(19) United States
(12) Patent Application Publication
(10) Pub. No.: US 2024/0016625 A1
(43) Pub. Date: Jan. 18, 2024

(54) IMPROVEMENTS IN AND RELATING TO SURGICAL INSTRUMENTS, SYSTEMS AND METHODS

(71) Applicant: DEPUY IRELAND UNLIMITED COMPANY, RINGASKIDDY, CO CORK (IE)

(72) Inventors: ALEC BIRKBECK, LEEDS (GB); JEREMY FONSECA, RAMSBOTTOM (GB); JOHN SHAPLAND, SHEFFIELD (GB); NIKI GOSLING, LEEDS (GB)

(21) Appl. No.: 18/254,239

(22) PCT Filed: Dec. 21, 2021
(86) PCT No.: PCT/EP2021/086965

§ 371 Date: May 24, 2023

(30) Foreign Application Priority Data
Dec. 21, 2020 (GB) 2020305.5

(51) Int. Cl. A61F 2/46

(52) U.S. Cl. CPC A61F 2/4603; A61F 2002/4619

(57) ABSTRACT

A surgical instrument (100) and system incorporating such instruments, the instrument comprising: a stem (112), the stem having a proximal end (116) and having a distal end (110); a handle portion (114), the handle portion being provided towards the proximal end of the stem; and a guide element (108), the guide element being provided towards the distal end of the stem, wherein the guide element provides: a body element (131) defining a guide channel (128), the guide channel having a longitudinal axis (X-X) and being open at both ends along the longitudinal axis; and one or more positioning elements (130a, 130b). The instrument and system are suited to the removal of a polyethylene acetabular liner (25) from an implanted shell (27), for instance to remove that liner and replace it with another, whilst minimising the risk of damage to the shell holding it.

### TECHNICAL FIELD

The disclosure is concerned with the removal of a liner from an acetabular shell implant, for instance of the type used in hip arthroplasty. The disclosure includes surgical instruments for the removal of such liners, systems for the removal of such liners and methods of use for the surgical instruments and/or systems.

### BACKGROUND

In hip arthroplasty, the anatomic reconstruction of the joint is sought. The natural acetabulum is removed and replaced with an implant formed of an acetabular shell placed in the recess in the bone and fixed in position. Subsequently, an appropriately sized liner is inserted into the shell, mated and locked. On occasions, it is necessary to remove a polyethylene acetabular liner from an implanted shell, for instance to remove that liner and replace it with another.

Existing designs provide for jaw based surgical instruments which are used to take hold of the liner and apply a force to pull the liner from the shell.

It is desirable for the surgical instrument, system or methods of use to remove the liner without damaging or risking damage to the shell holding it. It is desirable for the surgical instrument, system or methods of use to be easily positioned, used and removed. It is desirable for the surgical instrument, system or method of use to effectively and reliably remove the liner, in wound sites which are hard to access or have limited access.

### SUMMARY

According to a first aspect of the invention there is provided a surgical instrument, the instrument comprising:
a stem, the stem having a proximal end and having a distal end;
a handle portion, the handle portion being provided towards the proximal end of the stem; and
a guide element, the guide element being provided towards the distal end of the stem, wherein the guide element provides:
   a body element defining a guide channel, the guide channel having a longitudinal axis and being open at both ends along the longitudinal axis; and
   one or more positioning elements.

The guide channel may be completely surrounded by the body element. The guide channel may be a through bore in the body element.

The guide channel may be only partially surrounded by the body element. For instance, the guide channel may be surrounded by the body element for greater than 190° about the longitudinal axis, optionally for greater than 200° and more optionally for greater than 220°.

The guide channel may, in use receive a cutting tool, for instance a drill bit. The longitudinal axis of the guide channel may correspond to the operative axis of the cutting tool in use, optionally the rotational axis of the cutting tool. The guide channel may, in use, restrain radial movement of the cutting tool, for instance relative to the longitudinal axis of the guide channel and/or relative to the operative axis of the cutting tool. The guide channel may, in use, allow axial movement of the cutting tool relative to the guide channel, for instance along the longitudinal axis of the guide channel in one or both directions and/or along the operative axis of the cutting tool in one or both directions.

The guide channel may define the angularity of a hole formed in a liner by a cutting tool. The angularity may be configured such that the hole formed in the liner is spaced from a retention mechanism for the liner in the shell. The angularity may be configured such that the hole is formed closer to the centre of the liner and/or shell than the retention mechanism.

The guide channel may define the position of the hole formed in a liner by a cutting tool. The position may be configured such that the hole formed in the liner is spaced from a retention mechanism for the liner in the shell. The position may be configured such that the hole is formed closer to the centre of the liner and/or shell than the retention mechanism.

The body element may completely surround the guide channel. The body element may only partially surround the guide channel. The body element may have a through bore, particularly a right cylinder through bore. The through bore may have a longitudinal axis which is inclined relative to a superior face of the body element and/or relative to an inferior face of the body element. The through bore may have a longitudinal axis which is perpendicular to a superior face of the body element and/or perpendicular to an inferior face of the body element. The body element may be at least partially annular about the longitudinal axis.

The guide element may comprise the body element and the one or more positioning elements. The guide element may further comprise a base element. The guide element may consist of the body element, a base element and the one or more positioning elements.

The one or more positioning elements may extend from the guide element, particularly a base element provided by the guide element.

The base element may be provided intermediate the body portion and a part of the stem. The base element may provide a mount for the body portion, for instance relative to the stem. The base element may be mounted on the stem, particularly the transition section of a stem, optionally the distal end of the transition section. The base element may be curved about the longitudinal axis.

The one or more positioning elements may be adapted to provide a visual guide to correct positioning and/or correct alignment of the guide channel, for instance relative to, in use a liner. The one or more positioning elements may be adapted to provide a haptic guide to correct positioning and/or correct alignment of the guide channel, for instance relative to, in use, a liner.

The one or more positioning elements may be provided by one or more positioning location supports.

In an embodiment, one positioning element may be provided. The one positioning element may be a curved element. The one positioning element may include an edge. The one positioning element may be provided by one or more positioning location supports, for instance a pair of positioning location supports that extend to either side of a guide element. Two or more positioning location supports may provide a contiguous positioning element.

In an alternative embodiment, two or more positioning elements may be provided. Two positioning elements may be provided. One or more pairs of positioning elements may be provided. One half of one or more or all of the pairs of positioning elements may be provided by a first positioning location support and the other half by a second positioning location support. The second positioning location support may be a mirror image of a first positioning location support.

A first positioning location support may be provided which extends in a first direction. The first direction may be away from the longitudinal axis to one side thereof. The first direction may be away from the guide element and/or body element and/or base element, to one side thereof. The first positioning location support may extend in a first further direction. The first further direction may be parallel to the longitudinal axis and distally. The first further direction may be away from the guide element and/or body element and/or base element, in a distal direction.

The first positioning location support may have a proximal end. The proximal end may lead to or connect to the base element. The first positioning location support may have a distal end. The distal end may be distal relative to the proximal end of the first positioning location support and/or may be further distally than the stem and/or the base element and/or the body element. The distal end may be the most distal part of the surgical instrument, potentially equal with the second positioning location support's distal end.

The first positioning location support may be curved. The first positioning location support may be curved away from the stem, particularly the transition section of the stem.

A second positioning location support may be provided which extends in a second direction. The second direction may be away from the longitudinal axis to a second side thereof, optionally to the other side relative to the first positioning location support. The second direction may be away from the guide element and/or body element and/or base element, to a second side thereof, optionally to the other side relative to the first positioning location support. The second positioning location support may extend in a second further direction. The second further direction may be parallel to the longitudinal axis and distally. The second further direction may be away from the guide element and/or body element and/or base element, in a distal direction.

The second positioning location support may have a proximal end. The proximal end may lead or connect to the base element. The second positioning location support may have a distal end. The distal end may be distal relative to the proximal end of the second positioning location support and/or may be further distally than the stem and/or the base element and/or the body element. The distal end may be the most distal part of the surgical instrument, potentially equal with the first positioning location support's distal end.

The second positioning location support may be curved. The second positioning location support may be curved away from the stem, particularly the transition section of the stem.

An axis of symmetry may exist between the first positioning location support and the second positioning location support extending parallel to the longitudinal axis.

The distal end of the first positioning location support and the distal end of the second positioning location support may define a chord extending between them. The chord may be closer to the longitudinal axis of the guide channel than a chord drawn between a first other part of the first positioning location support and a first other part of the second positioning location support, optionally between any other part of the first positioning location support and any other part of the second positioning location support. The chord may be closer to the longitudinal axis of the guide channel than a chord drawn between the proximal end of the first positioning location support and the proximal end of the second positioning location support.

One or more positioning locations may be provided by the one or more positioning elements. One or more or all of the positioning locations may be those locations which abut the liner in use.

In an embodiment, one positioning location is provided by one or more positioning elements. The one positioning location may be provided by a curved part of one or more positioning elements. The one positioning location may be provided by an edge of one or more positioning elements. Optionally the one positioning location is provided by two positioning elements,

In an alternative embodiment, two or more separate positioning locations may be provided by one or more positioning elements. The two or more positioning locations may be provided by different curved parts of two or more positioning elements, optionally different parts of the same curve. The two or more positioning locations may be provided by different edges of two or more positioning elements, optionally different parts of the same edge. Optionally the positioning locations may be provided by two or more positioning elements.

One or both or all the positioning elements may be provided with one or more liner engaging elements. One or both or all of the positioning locations may be provided with liner engaging elements. The liner engaging elements may be provided at the ends of the positioning element[s] or adjacent thereto. The liner engaging elements may be provided at the ends of the positioning location[s] or adjacent thereto.

One or more or all of the positioning elements may include one or more liner penetrating parts. One or more or all of the positioning locations may include one or more liner penetrating parts. One or more or all of the liner engaging elements may include one or more liner penetrating parts. The liner penetrating parts may be provided at the ends of the positioning element[s] or adjacent thereto. The liner penetrating parts may be provided at the ends of the positioning location[s] or adjacent thereto.

One or more or all of the liner engaging elements may include a curved element, for instance a curved edge. One or more or all of the liner engaging elements may include an edge. One or more or all of the curved element[s] and/or edge[s] may be provided towards or at the distal end of their positioning element. One or more or all of the of the curved element[s] and/or edge[s] may project away from the guide element, particularly away from the base element. One or more or all of the of the curved element[s] and/or edge[s] may project in the general direction of the longitudinal axis. One or more or all of the of the curved element[s] and/or edge[s] may, in use, extend towards the liner. One or more or all of the of the curved element[s] and/or edge[s] may extend, in use, towards the perimeter edge wall of the liner at the junction of the liner and the shell. One or more or all of the of the curved element[s] and/or edge[s] may abut or extend from a wider part, for instance the base element, so as to limit the depth of penetration of the teeth into the liner, in use.

One or more or all of the liner engaging elements may be teeth. Optionally two teeth may be provided on each positioning element. One or more or all of the teeth may be provided towards or at the distal end of their positioning element. The one or more or all of the teeth may be provided by the ends of an edge, for instance a curved edge. One or more or all of the teeth may project away from the guide element, particularly away from the base element. One or more or all of the teeth may project in the general direction of the longitudinal axis. One or more or all of the teeth may, in use, extend towards the liner. One or more or all of the teeth may extend, in use, towards the perimeter edge wall of the liner at the junction of the liner and the shell. One or more or all of the teeth may abut or extend from a wider part, for instance the base element, so as to limit the depth of penetration of the teeth into the liner, in use.

One or more secondary positioning locations may be provided by the one or more positioning elements. One or more of the secondary positioning locations may abut the liner, in use. One or more secondary positioning locations may be provided by the guide element. One or more secondary positioning locations may be provided by the body element. One or more secondary positioning locations may be provided by the distal end face of the body element. The distal end face may be planar. One or more secondary positioning locations may abut the superior face of the liner in use and/or the superior face of the shell.

The guide element may be provided at the distal end of the stem.

The guide element may be connected to a first section of the stem. The guide element may be connected to a transition section of the stem. The transition section may extend away from the guide element, optionally in a direction having a radial component relative to the longitudinal axis. The radial component may be the major component of the directional extent of the transition section. The transition section may provide a gap or spacing between the longitudinal axis and one or more or all parts of the stem and/or handle portion.

The stem may include a second section. The second section may be connected to the first section. The stem may include an aligned section. The aligned section may extend in a direction having a parallel component to the longitudinal axis. The parallel component may be the major component of the directional extent of the aligned section. The aligned section may substantially maintain the gap or spacing between the longitudinal axis and one or more or all parts of the stem and/or handle portion. The aligned section may not substantially increase or decrease the gap or spacing between the longitudinal axis and one or more or all parts of the stem and/or handle portion.

The stem may include a third section. The third section may be connected to the second section. The stem may include an inclined section. The inclined section may extend in a direction having a parallel component to the longitudinal axis and having a radial component relative to the longitudinal axis. The parallel component may be the dominant component of the directional extent of the inclined section. The inclined section may have an angle of intersection with the longitudinal axis of between 20° and 45°, for instance between 25° and 35°. The inclined section may increase the gap or spacing between the longitudinal axis and one or more or all parts of the stem and/or handle portion. The inclined section may not substantially decrease the gap or spacing between the longitudinal axis and one or more or all parts of the stem and/or handle portion.

One or more or all sections of the stem may be configured to provide a line of sight between the stem and the longitudinal axis towards the guide element and optionally to the guide element, for instance to the one or more positioning elements.

The handle portion may be provided at the proximal end of the stem. The handle portion may be configured to assist with gripping of the handle. The handle may be orientated and/or configured and/or positioned to maintain the hand of the user out of the line of sight towards the guide element.

The handle portion may be configured to enable the user to apply axial force along and/or parallel to the longitudinal axis, particularly to the guide element. The handle portion may be configured to enable the user to apply radial force, particularly to the guide element.

The first aspect of the invention may include any of the other features, options or possibilities set out herein, including in the other aspects of the invention, and including when any are taken singularly or in any combination.

According to a second aspect of the invention there is provided a surgical system, the surgical system comprising:
a surgical instrument, the instrument comprising:
   a stem, the stem having a proximal end and having a distal end;
   a handle portion, the handle portion being provided towards the proximal end of the stem; and
   a guide element, the guide element being provided towards the distal end of the stem, wherein the guide element providing:
      a body element defining a guide channel, the guide channel having a longitudinal axis and being open at both ends along the longitudinal axis; and
   one or more positioning elements; and
      further comprising:
one or more cutting tools; and
one or more elongate at least partially threaded elements.

The surgical instrument may be provided according to the first aspect of the invention and/or include any of the other features, options or possibilities set out herein, including in the other aspects of the invention, and including when any are taken singularly or in any combination.

The surgical systems may further comprise an actuator, particularly for application of torque to the cutting tool, in use. The actuator may act on the cutting tool directly or may be connected to one or more intermediate elements, such as a drive extension. The actuator may be manual or may be powered.

The one or more cutting tools may be or may include a drill bit. The one or more cutting tools may be provided discrete from the surgical instrument. The one or more cutting tools may have a distal section provided with one or more cutting blades. The one or more cutting tools may have a proximal end with an engagement for an actuator or an intermediate element. The engagement may provide for the transfer of torque to the cutting tool. The engagement may provide for the transfer of axial force, in a distal direction, to the cutting tool.

The guide channel of the surgical instrument may be configured to receive a cutting tool, for instance a drill bit, with the cross-section of the guide channel being the cross-section of the cutting tool plus a clearance. The longitudinal axis of the guide channel may correspond to the operative axis of the cutting tool in use, optionally the rotational axis of the cutting tool.

The cutting tool may be introduced to the guide channel at the superior side of the body element. The cutting tool may pass through the guide channel to extend beyond the inferior side of the body element. The cutting tool may provide the cutting action extending from the inferior aside of the body element. The cutting tool may be retracted into and out of the guide channel after use.

The guide channel may define the angularity of a hole formed in a liner by a cutting tool.

One or more or all of the elongate at least partially threaded elements may be provided with a screw thread, particularly a self-tapping screw thread. The one or more elongate elements may be provided discrete from the surgical instrument. The one or more elongate elements may have a distal section provided with one or more threads. The one or more elongate elements may have a proximal end with an engagement for an actuator or an intermediate element. The engagement may provide for the transfer of torque to the elongate element. The engagement may provide for the transfer of axial force, in a distal direction, to the elongate element.

The cutting tool engagement and/or elongate element engagement may have any of the following further options, features and possibilities set out in the following separate aspect of the disclosure.

The second aspect of the invention may include any of the other feature features, options or possibilities set out herein, including in the other aspects of the invention, and including when any are taken singularly or in any combination.

According to a third aspect of the invention there is provided an engagement system for a surgical component and an actuator system for the surgical tool, the engagement comprising:
an internal bore in the distal end of an element of the actuator system;
a male section on the proximal end of the surgical tool adapted to be received within the internal bore;
   wherein the male section includes one or more engagement surfaces for cooperation with one or more engagement surfaces included in the internal bore to provide torque transmission from the actuator system to the surgical tool; and
   wherein the male section being in the internal bore compresses a resilient element between a part of the male section and a part of the internal bore to provide a level of resistance to axial movement of the male section out of the internal bore.

The surgical component may be a surgical tool. The surgical tool may be a cutting tool or for instance a drill bit. The surgical component may be a surgical tool such as an at least partially threaded elongate element, for instance for separating a liner and shell.

The actuator system may include a manual or powered actuator. The actuator system may include one or more intermediate elements provided between the actuator and the surgical tool, such as a drive extension. Optionally the surgical tool is mounted on the drive extension.

The internal bore may be in the distal end of the actuator or may be in the distal end of an intermediate element, such as a drive extension.

The male section may be provided with one or more exterior engagement surfaces. The internal bore may be provided with one or more interior engagement surfaces, optionally with a profile engaging with at least a portion of the one or more exterior engagement surfaces of the male component. The male section exterior engagement surfaces may correspond to the internal bore engagement surfaces minus a tolerance. A hexagonal cross-sectional profile may apply to the exterior engagement surfaces and/or interior engagement surfaces.

The internal bore may have a depth defined by a base section. Optionally the proximal end of the surgical tool, in use, may abut the base section. Optionally the abutment constrains axial movement of the surgical tool into the bore and/or allows application of axial force to encourage the surgical tool into the material of the liner.

The resilient element may be an O-ring. The O-ring may be retained within a groove provided on the surgical tool. The O-ring may have a radial extent beyond the limits of the groove to provide the compressibility. The O-ring may be opposed by an annular surface in the internal bore. The O-ring may have a first non-compressed state in which its maximum radius or maximum profile exceeds the minimum radius or minimum profile of the opposing surface of the internal bore. The O-ring may have a second compressed state in which its maximum radius or maximum profile is reduced by the opposing surface of the internal bore.

The resilient element may be retained on the male section. The resilient element may be retained at a location closer to the proximal end than the engagement surfaces. The resilient element may be retained at a location further from the proximal end than the engagement surfaces.

The resilient element may be provided between a part of the male section and a part of the internal bore

The level of resistance to axial movement of the male section out of the internal bore may be above the level needed to retain the proximal end of the surgical tool in the internal bore during movement to the position of use and/or during their movement away from the position of use and/or during insertion through the guide channel to the liner. The level of resistance to axial movement of the male section out of the internal bore may be above the level needed to prevent the weight of the surgical tool or an impact on the surgical tool causing the surgical tool to detach from the internal bore.

The level of resistance to axial movement may be below the level at which an operator would encounter difficulty in removing the surgical tool from the internal bore.

The third aspect of the invention may include any of the other feature features, options or possibilities set out herein, including in the other aspects of the invention, and including when any are taken singularly or in any combination.

According to a fourth aspect of the invention there is provided a method for removing a liner from an acetabular shell, the method comprising:
providing a surgical instrument, the instrument comprising:
   a stem, the stem having a proximal end and having a distal end;
   a handle portion, the handle portion being provided towards the proximal end of the stem; and
   a guide element, the guide element being provided towards the distal end of the stem, wherein the guide element provides:
      a body element defining a guide channel, the guide channel having a longitudinal axis and being open at both ends along the longitudinal axis; and
      one or more positioning elements;
positioning the one or more positioning elements on a face of the liner and/or shell;
introducing a cutting tool to the liner, the cutting tool being passed through the guide channel;
cutting a hole in the liner using the cutting tool.

The method may further include introducing an elongate partially threaded element to the hole in the liner and optionally y tapping the threaded element into the liner to push the liner away from the shell.

The method may include introducing the instrument to the liner and/or shell and then adjusting the position to give the positioning of the one or more positioning elements. The method may include introducing one or more of the one or more positioning elements onto a superior face of the liner and/or onto a superior face of the shell. The method may include introducing the instrument into proximity with a junction between the shell and the liner. The method may include adjusting the position to bring the one or more positioning elements into contact with a part of the junction, for instance the superior face of the shell and the side wall of the liner extending superior to the superior face of the shell. The distal ends of the one or more position elements may be introduced and/or positioned in this way.

The method may include introducing the distal end[s] of the one or more positioning elements to the superior face of the shell to limit generally axial movement of the instrument. The method may include adjusting the position by sliding the distal end[s] radially inward relative to the shell and towards the liner. The method may include positioning the positioning element[s] in abutment with the perimeter edge wall of the liner. The positioning may resist further inward movement of the distal ends of the positioning elements.

The method may include defining the axis of the hole in the liner using the longitudinal axis of the guide channel.

The method may include positioning the one or more positioning elements in abutment with the outside of the liner and then causing one or more parts of the guide element to penetrate the material of the liner and give the final positioning. One or more positioning elements may penetrate the liner. One or more positioning locations may penetrate the liner. One or more liner engaging elements may penetrate the liner. One or more teeth may penetrate the liner.

The application of radial force to the guide element through the handle portion may cause penetration of the material of the liner and particularly penetration of the perimeter edge wall. The limited extent of the teeth and/or the inability of the parts of the guide element adjacent the teeth to penetrate the liner may finalise the radial position of the positioning elements, optionally of the guide element and ideally of the guide channel.

The method may include introducing a cutting tool to an actuator or an intermediate element, such as a drive extension.

The method may include introducing the cutting tool to the liner by one of more of: introducing the cutting tool to the guide channel at the superior side of the body element and/or passing the cutting tool through the guide channel to extend beyond the inferior side of the body element and/or axially advancing the cutting tool into contact with the liner.

The method may include cutting the hole with the cutting tool extending from the inferior aside of the body element.

The method may include rotating the cutting tool, for instance using a manual or using a powered actuator.

The method may include the guide channel restraining radial movement of the cutting tool, for instance relative to the longitudinal axis of the guide channel and/or relative to the operative axis of the cutting tool. The method may include the guide channel allowing axial movement of the cutting tool relative to the guide channel, for instance along the longitudinal axis of the guide channel in one or both directions and/or along the operative axis of the cutting tool in one or both directions.

The method may include, for instance once the desired depth of hole is formed, the retraction of the cutting tool. The method may include, for instance once the desired depth of hole is formed and/or once the cutting tool has been retracted from the guide channel, disengaging the surgical instrument from the liner and/or removing the surgical instrument from proximity with the liner and/or removing one or more parts of the surgical instrument from within the material of the liner.

The method may include repeating one or more or all of the steps of provide, introducing, positioning, cutting and removing at a plurality of locations to provide other holes. The other holes may be spaced around the perimeter of the liner.

The method may include the use of the same instrument and/or guide channel and/or cutting tool size and/or elongate threaded element size for a range of sizes and/or thicknesses of liner.

The method may include introducing an elongate partially threaded element to an actuator or an intermediate element, such as a drive extension. The actuator and/or intermediate element may be the same as previously attached to the cutting tool.

The method may further include introducing the distal end an elongate partially threaded element to the hole in the liner. The threaded element may be self-tapping. The method may include rotating the threaded element to advance the threaded element into the liner. The method may include advancing the threaded element through the liner and into contact with the shell. The method may include further rotation, but optionally with no further advancing, of the threaded element to cause the liner to be displaced from the shell towards the surgical instrument.

The fourth aspect of the invention may include any of the other feature features, options or possibilities set out herein, including in the other aspects of the invention, and including when any are taken singularly or in any combination.

### BRIEF DESCRIPTION OF THE DRAWINGS

Various embodiments of the disclosure will now be described, by way of example only, and with reference to the accompanying drawings in which:

FIG. 1a is a side view of a prior art liner extraction instrument;

FIG. 1b is a perspective view of the instrument of FIG. 1a engaging a liner;

FIG. 1c is a side view corresponding to FIG. 1b;

FIG. 1d is a side view with the liner partially removed from the shell;

FIG. 2 is a perspective view of a system according to the disclosure, including a guide instrument, drill bit, screw and drive extension;

FIG. 3 is a superior plan view of the distal end of the guide instrument and sectioned drill bit engaged with a liner in a shell;

FIG. 4 is a side view corresponding to FIG. 3;

FIG. 5 is a perspective view of details of a distal end of the drive extension, proximal end of a drill bit and proximal end of a screw according to another embodiment of the disclosure;

FIG. 6 is a side view showing the interaction with a first liner and shell combination;

FIG. 7 is a side view showing the interaction with a second liner and shell combination;

FIG. 8 is a detailed side perspective view of the guide piece with a drill bit present; and

FIG. 9 shows the pilot hole relative to a liner and shell.

### DETAILED DESCRIPTION OF THE DRAWINGS

In hip arthroplasty the anatomic reconstruction of the joint is sought. The natural acetabulum is removed and replaced with an implant formed of an acetabular shell placed in the recess in the bone and fixed in position. Subsequently, an appropriate sized liner is inserted into the shell, mated and locked. On occasions, it is necessary to remove a polyethylene acetabular liner from an implanted shell, for instance to remove that liner and replace it with another. This must be done whilst minimising the risk of damage to the shell holding it.

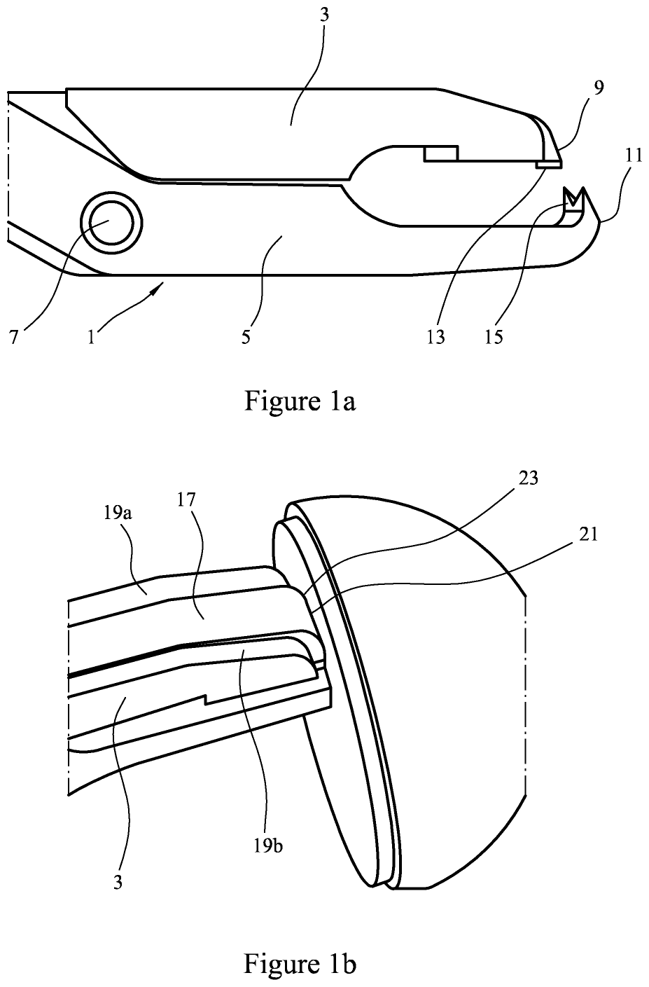

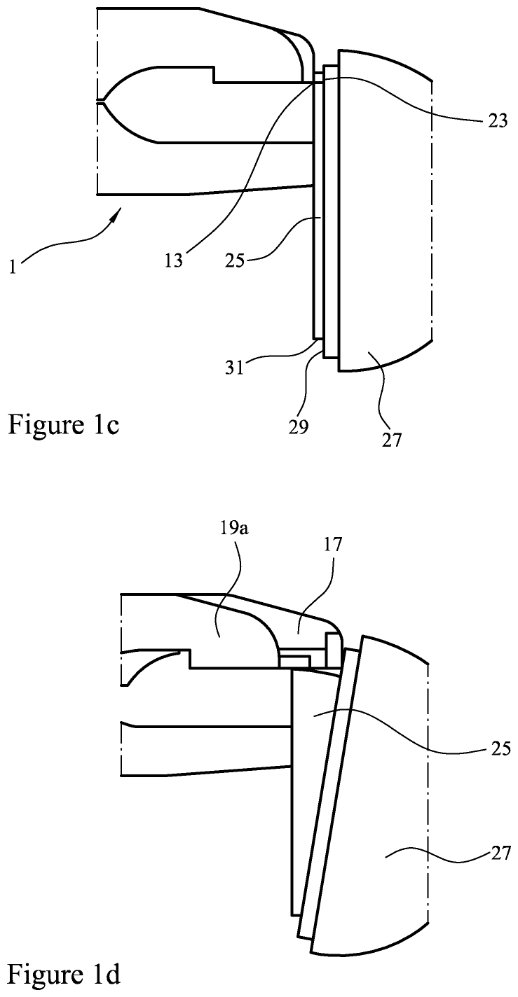

One prior art instrument, a liner extractor 1, is illustrated in FIG. 1a. This provides a first jaw 3 and second jaw 5 pivotally mounted relative to one another about pin 7. The distal end 9 of the first jaw 3 extends beyond the distal end 11 of the second jaw 5. The distal end 9 of the first jaw 3 is provided with a pair of contact surfaces 13 facing the second jaw 5. The distal end 11 of the second jaw 5 is provided with a pair of teeth 15 facing generally towards the first jaw 3.

As can be seen in FIG. 1b, the first jaw 3 has a separate central element 17 which is provided between a first jaw element 19a and second jaw element 19b which in combination form the first jaw 3. The distal end 21 of the central element 17 has an abutment surface 23 generally perpendicular to the pair of contact surfaces 13.

With the first jaw 3 and second jaw 5 open, wider than is shown in FIG. 1a where the closed state is shown, the liner extractor 1 can be engaged with a liner 25 sitting in a shell 27, as shown in FIG. 1c. The abutment surface 23 is brought into contact with the circumferential face 29 of the shell 27. This limits axial movement of the liner extractor 1 relative to the shell 27. The pair of contact surfaces 13 are brought into abutment with the small side wall 31 of the liner 25. Closing the jaws, by moving the second jaw 5 inwards towards the first jaw 3, brings the pair of teeth 15 into contact with the inside of the liner 25. The teeth 15 are sharp enough to penetrate the liner 25 upon application of force through the liner extractor 1, as the contact surfaces 13 and side wall 31 combine to prevent movement of the first jaw 3.

In FIG. 1d, the partial removal of the liner 25 from the shell 27 is shown. This is achieved by retraction of the first jaw element 19a and second jaw element 19b relative to the central element 17. Hence, the central element 17 maintains the position of the shell 27 and the liner 25 is eased out of the shell 27.

As access to the wound site is not easy, the positioning of the liner extractor 1 with the abutment surface 23 and contact surfaces 13 in the necessary position during movement of the contact surfaces and abutment surface relative to one another can be awkward.

Another instrument-based option for liner 25 removal is to form a pilot hole in the liner 25. Initial engagement of a self-tapping screw in the pilot hole is then provided, with tightened of the screw pushing between the liner 25 and the shell 27 with sufficient force to remove the liner 25 from the shell 27. Where the liner 25 is of the thicker and/or larger and/or stiffer variety, then multiple pilot holes and self-tapping screws may be needed.

In this type of option, to be effective, the pilot hole needs to be created in a consistent and precise location and also with a consistent angularity with respect to the liner 25. This needs to be achieved in a wound space which frequently only offers difficult access.

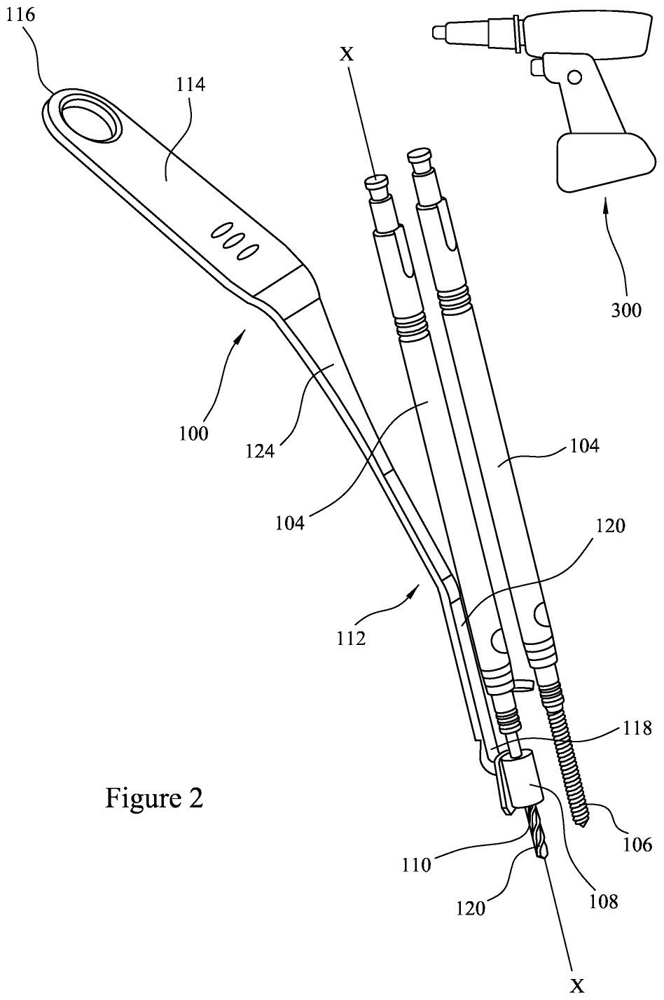

FIG. 2 illustrates one embodiment of the disclosure which provides a surgical instrument in the form of guide instrument 100, a surgical tool in the form of a drill bit 102, an intermediate section in the form of an extension drive 104. FIG. 2 also shows the same extension drive 104 engaged with a different surgical tool, a self-tapping screw 106.

The guide instrument 100 includes a guide element in the form of a guide piece 108 at the distal end 110. The guide piece 108 is mounted on a stem 112 with a handle portion 114 at the proximal end 116. The stem 112 is divided into several sections. A first section, a transition section 118, spaces the rest of the guide instrument 100 from the operative axis of the drill bit 102. A second section, aligned section 120, is aligned with but spaced from the operative axis of the drill bit 102 and thereby aligns force applied through the handle portion 114 to the liner 25 in use, with the axis along which force applied through the drill bit 102 passes to the liner 25 in use. A third section, inclined section 124, is inclined away from the operative axis of the drill bit 102 so as to increase the space and improve the line of sight for the surgeon down to the distal end 110. The third section, inclined section 124, leads to the handle portion 114.

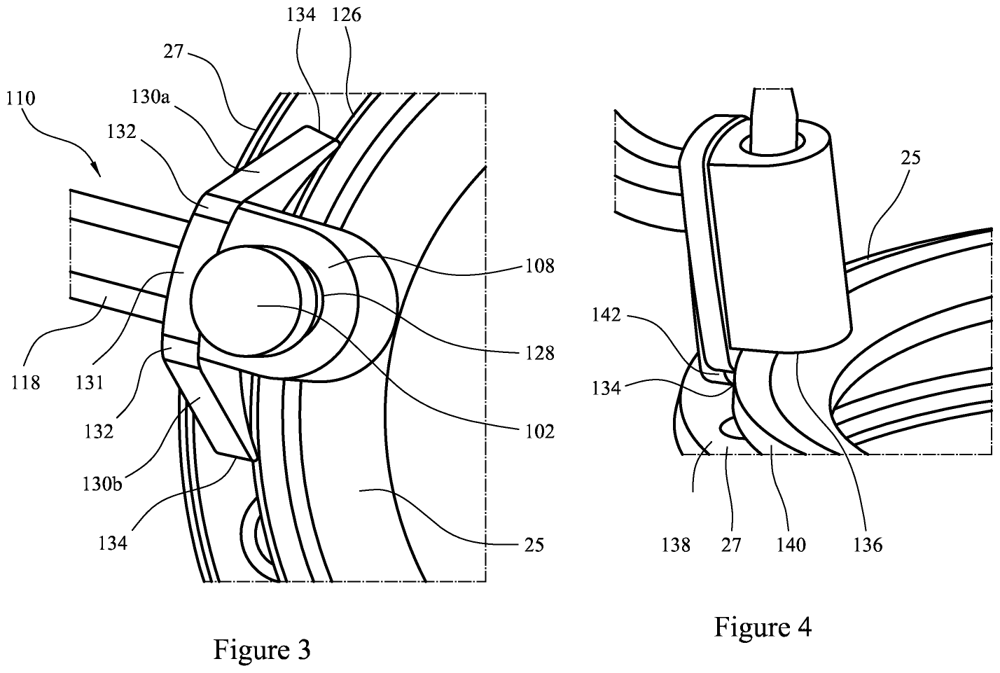

In use, as shown in FIG. 3, the distal end 110 of the guide instrument 100 is brought into proximity with the junction 126 between the liner 25 and the shell 27. The guide piece 108 on the distal end 110 provides a guide channel in the form of a through bore 128 through which the drill bit 102 can be inserted. The axis of the bore 128 matches the operative axis for the drill bit 102, in use.

The guide piece 108 is provided with a configuration that provides visual clues and guides to its correct alignment/positioning with respect to a liner 25 and/or shell 27. The guide piece 108 includes a first extending element 130a which extends from a body element 131, provided at the junction of the guide piece 108 and the transition section 118. The first extending element 130a extends in a first direction. A second extending element 130b is provided in the same manner, but extending in the opposing direction. The first and second extending elements are one form of the positioning location supports. Both the first extending element 130a and the second extending element 130b have a body element contacting end 132 and an extending element protruding end 137. The two protruding ends 137 are turned towards one another. A chord drawn between the two protruding ends 137 will pass closer to the axis of the bore 128 than a chord drawn between other parts of the extending elements and particularly between the body element contacting ends 132. In the illustrated example this is provided by curvature of the first extending element 130a and the second extending element 130b.

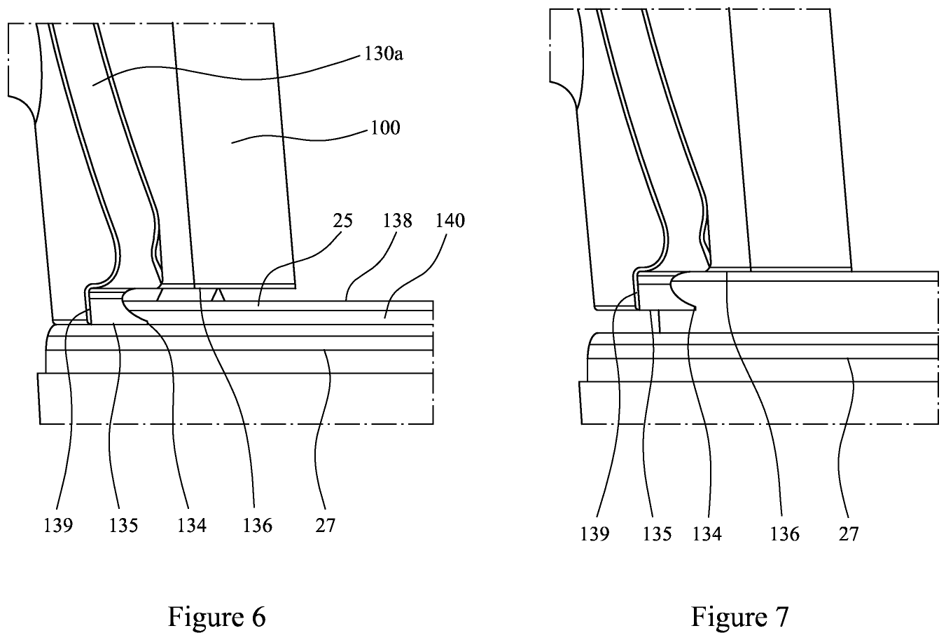

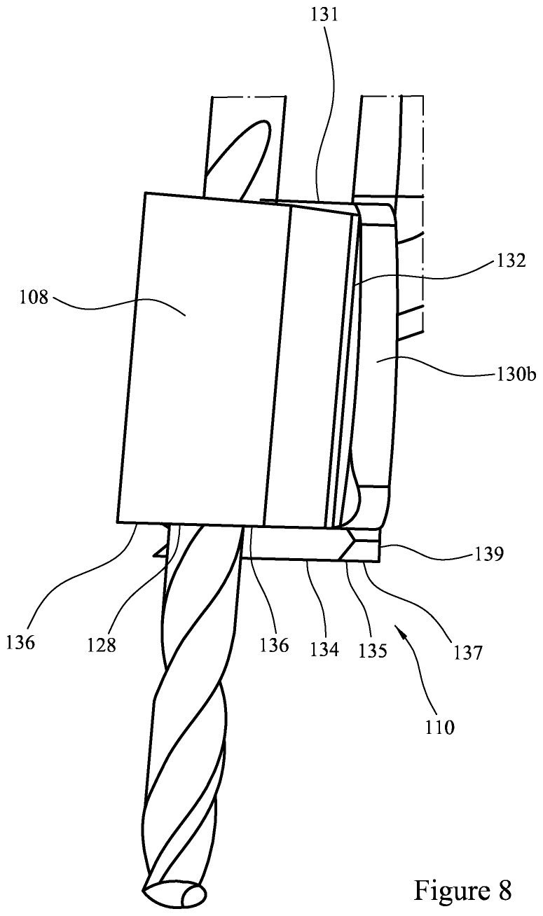

At the distal end 110 of the guide piece 108, as seen in FIGS. 6, 7 and 8, a planar distal end face 136 is provided around the bore 128. This is one form of the secondary positioning locations. In this example, a common distal end is provided for both the first extending element 130a and the second extending element 130b by a contiguous planar distal end face 135. This distal end face 135 extends the full length of the curvature of both the first extending element 30a and the second extending element 30b. This is one form or the positioning locations. The distal end face 135 is provided on a distal end section 139. The distal end section 139 has a smaller cross-section away from the distal end face 135 than at the distal end face 135 and hence a distal edge 134 is defined. The distal edge 134 extends along the full curvature of both the first extending element 30a and the second extending element 30b in this form.

Referring to FIG. 6, the interaction of the distal edge 134 with a first combination of shell 27 and liner 25 is shown. In this example, the liner 25 has a relatively small extent of protrusion from the shell 27 and so the perimeter edge wall 140 has only a small height. Different interactions, as further exemplified below, will occur for different liner 25 and shell 27 combinations.

In FIG. 6, the distal end face 135 and distal edge 134 has a greater distal extent relative to the guide instrument 100 than the guide piece 108, for instance the distal end face 136 thereof. The planar distal end face 135 is parallel to but not co-planar with the distal end face 136 of the guide piece 108.

In use, this means that as the guide instrument 100 is brought towards the shell 27 and liner 25, one end then the other or both simultaneously of the distal end face 135 will abut the circumferential end face 138 of the shell 27. This limits generally axial movement of the guide instrument 100. The distal end face 135 can then be slid radially inward relative to the shell 27 and liner 25 across the end face 138. This continues until parts of the distal edge 134 abut the raised perimeter edge wall 140 of the liner 25 formed by the liner 25 extending slightly above the plane of the end face 138 of the shell 27. At this point, the liner 25 resists further inward movement. In the illustrated form, the curvature of the distal edge 134 is such that the ends of the distal edge 134 provide the abutment. The profile of the distal edge 134 is such that its ends act like teeth 142. The distal edge 134 faces laterally away from the extending elements relative to the proximal to distal orientation of those extending elements. That is the distal edge 134 faces towards the liner 25 in use, and in particular towards the perimeter edge wall 140 at the junction of the liner 25 and shell 27. The application of radial force to the guide piece 108 through the handle portion 114 causes the teeth 142 at the ends of the distal edge 134 to penetrate the material of the liner 25 and particularly to penetrate the perimeter edge wall 140. The limited extent of the distal edge 134 and it not being possible to cause the extending element behind them to penetrate the liner 25, means that the radial position of the extending elements 130a, 130b and hence of the guide piece 108 and hence the bore 128 is very closely controlled. The pilot hole is created in a consistent and precise location.

In an alternative format, the curvature of the distal edge 134, particularly its radius of curvature, could be greater than the curvature, particularly radius of curvature of the liner, such that intermediate locations on the distal edge 134 abut the liner. The distal edge 134 is sharp enough along its length to penetrate the liner 25 and so act in a tooth like manner at that abutment.

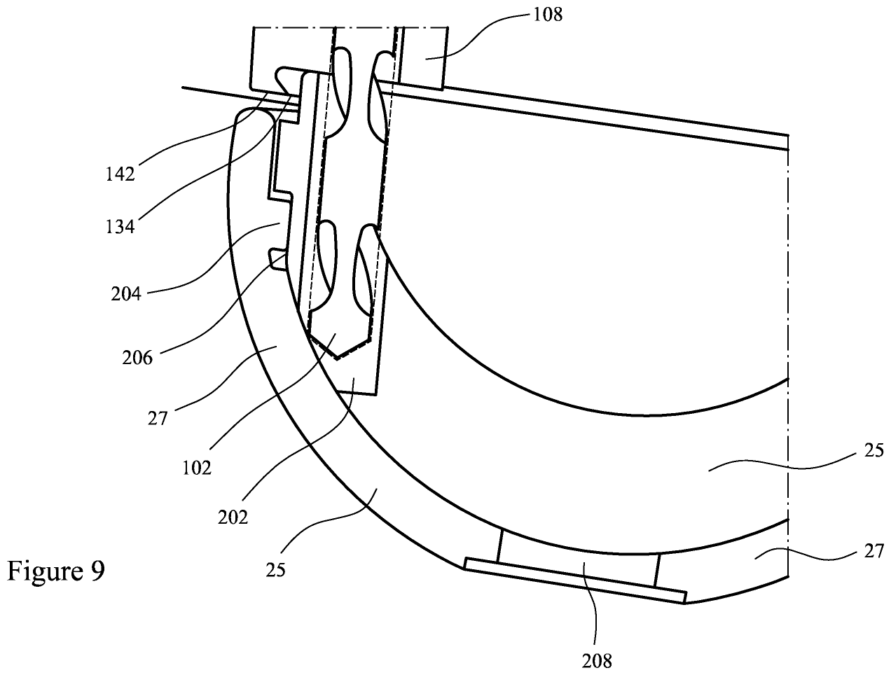

FIG. 9 shows the pilot hole 202 being formed by a drill bit 102. The drill bit 102 extends through the guide piece 108, into the liner 25 and down to the junction between the liner 25 and the shell 27. The teeth 142 at the ends of the curved edge 134 are engaged with the liner 25.

The location used for the pilot hole 202 is adjacent the peripheral edge of the liner 25 and is also adjacent the peripheral edge of the shell 27. In this area, no contact between the shell 27 and the liner 25 is intended in use, and so, even if the removal of the liner 25 by the disclosure causes minor damage to the shell 27, that minor damage is not in a material location on the shell 27. If a location further into the liner 25, for instance at or close to the pole of the liner 25 and shell 27, was used then any damage would be in a material area. The location used for the pilot hole 202 is however far enough radially inward relative to the edge of the liner 25, that the pilot hole 202 is away from the retaining mechanism 204, 206 for the liner 25 in the shell 27. In the illustrated example, a lug 204 on the shell 27 engages with a recess 206 on the liner 25, but other retention mechanisms are possible.

The peripheral location for the pilot hole 202 has been established in testing to offer the most effective approach to removing the liner 25 from the shell. This is particularly important in providing enough force to overcome the retention force between the liner 25 and the shell 27. The peripheral location is more effective than locations further into the liner 25, for instance at or close to the pole 208 of the liner 25 and shell 27.

Referring to FIG. 7, a different interaction of the distal edge 134 with a first combination of shell 27 and liner 25 is shown. In this example, the liner 25 has a relatively large extent of protrusion from the shell 27 and so the perimeter edge wall 140 has a significant height.

In use, this means that as the guide instrument 100 is brought towards the shell 27 and liner 25, the distal end face 136 will abut the circumferential end face 200 of the liner 25. This limits generally axial movement of the guide instrument 100. The distal edge 134 in this position is still spaced from the circumferential end face 138 of the shell 27, but the distal edge 134 can still be slid radially inward relative to the shell 27 and liner 25 until sections of the distal edge 134, in the illustrated example the teeth 142 at the ends, abut the raised perimeter edge wall 140 of the liner 25 formed by the liner 25 extending slightly above the plane of the end face 138 of the shell 27. At this point, the liner 25 resists further inward movement. The application of radial force to the guide piece 108 through the handle portion 114 causes the distal edge to penetrate the material of the liner 25 and particularly to penetrate the perimeter edge wall 140.

Whilst the embodiments above reference a tooth 142 at each end of a continuous distal edge 134 for providing the penetration of the liner 25, it is possible to provide two or more teeth in a other positions. A series of teeth spaced along the distal edge 134 and/or a serrated distal edge 134 could be provided. Equally it is also possible to rely upon intermediate parts of the distal edge 134 engaging with and penetrating the liner 25.

The configuration of the distal edge 134 of the extending elements and/or the teeth 142 means that a wide range of different diameter shells 27 and liners 25 can be used successfully with the same guide instrument 100.

The drill bit 102 is provided separately from the guide instrument 100 and is mounted on an extension drive or other actuating instrument 104, optionally in the manner described in more detail below. The same diameter of drill bit 102 is suitable for a wide range of liner 25 sizes and so a given guide piece 108 and guide instrument 100 is also widely applicable.

The guide piece 108 through bore 128 has an axis X-X which gives the desired pilot hole with a consistent angularity with respect to the liner 25 as the bore 28 limits the operative axis of the drill bit 102 to the same axis X-X. The cross-section of the bore 28 perpendicular to the axis X-X [plus a clearance tolerance] matches that of the cross-section of the drill bit 102 also perpendicular to the axis X-X.

In use, therefore, with the guide instrument 100 in position on the shell 27 and liner 25, with parts of the distal edge 134 or teeth 142 in the material of the liner 25, the drill bit 102 on the drive extension 104 is slid into the bore 128 and into abutment with the surface of the liner 25. A manual or powered actuator 300 [shown schematically in FIG. 2] connected to the drive extension 104 is used to rotate the drill bit 102 and hence form a pilot hole in the liner 25. Once the desired depth of pilot hole is formed, the drive extension 104 and the drill bit 102 can be retracted. The guide instrument 100 can then be moved outward to disengage the parts of the distal edge 134 or teeth 142 and can then also be withdrawn.

If necessary, the process can be repeated at other locations to provide other pilot holes.

A drive extension 104, which could be the same one with the drill bit 102 disconnected, is then provided with a self-tapping screw 106. The distal end of the screw 106 is inserted into the end of the pilot hole and rotation of the drive extension 104 causes the screw 106 to advance into the liner 25. Continued advancement of the screw 106 causes the liner 25 to be displaced from the shell 27.

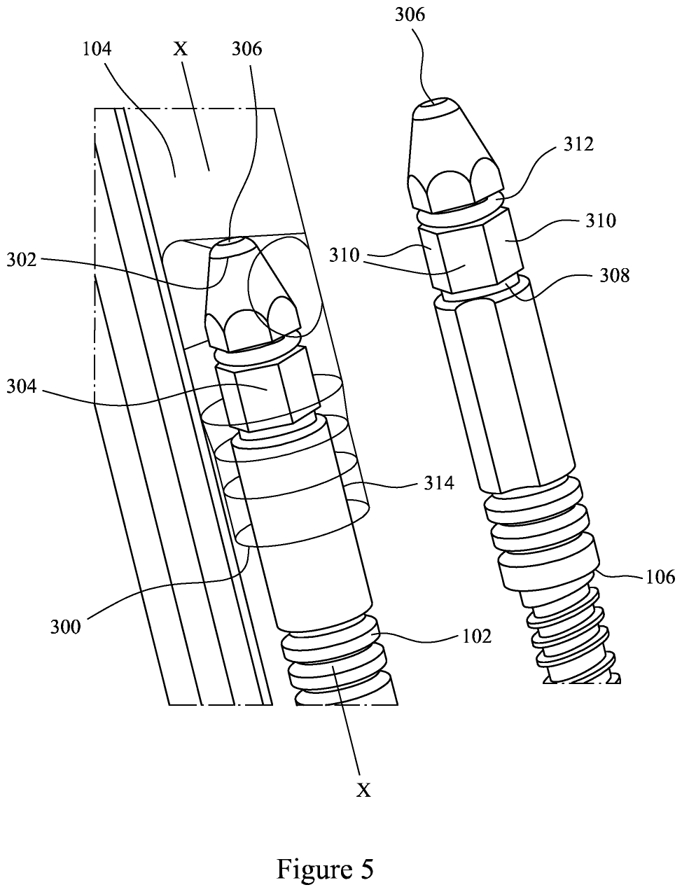

The drill bit 102 can be mounted on an extension drive 104 or other actuating instrument 104 in various ways. One option under the disclosure is illustrated in FIG. 5.

The drive extension 104 has an internal bore in its distal end 300 which receives the proximal end 302 and end section 304 of the drill bit 102 and similarly the proximal end 306 and end section 308 of the screw 106. The end sections 304, 308 have equivalent cross-sectional profiles and as illustrated are provided with six regularly spaced and matching size flats 310 which provide an hexagonal drive interface. The internal bore of the drive extension 104 is provided with a corresponding drive profile; in the illustrated case with six regularly spaced and matching size flats 310 which provide an hexagonal drive interface. The cooperation of the two drive faces readily transfers torque from the drive extension 104 to the drill bit 102 or screw 106. The depth of the internal bore, considered along axis X-X, matches the length of the end sections 304, 308 such that abutment of the proximal ends 302, 306 with the base 312 of the bore 314 results in correct alignment of the drive interfaces. The abutment also constrains axial movement of the drill bit 102 or screw 106 into the drive extension 104 and allows application of axial force to encourage the drill bit 102 or screw 106 into the material of the liner 25.

It is desirable to provide for easy engagement and disengagement of the drill bit 102 and/or screw 106 with the drive extension 104. At the same time, there is a need to retain the drill bit 102 and/or screw 106 on the drive extension 104 during their movement to the position of use and during their movement away from the position of use. In the disclosure, this level of axial restraint for the drill bit 102 and/or screw 106 against relative axial movement away from the drive extension 104 is provided by the interaction of an O-ring 312 on one with the surface on the other. In one form, the O-ring 312 is provided on the single use drill bit 102 rather than in the internal bore 314 of the drive extension 104. Similarly the O-ring 312 may be provided on the single use screw 106 rather than in the internal bore 314 of the drive extension 104.

In that one form, as the drill bit 102 is slid into the internal bore 314, at least when the proximal end 302 approaches the base 312 of the bore 314, the internal profile of the bore 314 causes compression of the O-ring 312. The resilience of the O-ring 312 resists this compression and hence resists axial movement of the drill bit 102 out of the bore 314. The resistance is sufficient to prevent the weight of the drill bit 102 or a knock to the drill bit 102 causing it to detach from the drive extension 104. The O-ring 312 on the screw 106 operates and interacts in an equivalent manner. The resistance to axial movement is limited, however, and so the drill bit 102 or screw 106 can readily be pulled out of the drive extension 104 when desired, for instance to swap from the drill bit 102 to a screw 106. As the drill bit 102 and screw 106 are intended to be single use, there is no need to sterilise a structure with an O-ring 312 present.

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

1. A surgical instrument, the instrument comprising:
a stem, the stem having a proximal end and having a distal end;
a handle portion, the handle portion being provided towards the proximal end of the stem; and
a guide element, the guide element being provided towards the distal end of the stem, wherein the guide element provides:
a body element defining a guide channel, the guide channel having a longitudinal axis and being open at both ends along the longitudinal axis; and
one or more positioning elements.

2. The instrument of claim 1, wherein the guide channel is adapted to receive a cutting tool and a longitudinal axis of the guide channel corresponds to the operative axis of the cutting tool in use.

3. The instrument of claim 2, wherein the guide channel is adapted to restrain radial movement of the cutting tool and allow axial movement of the cutting tool relative to the guide channel.

4. The instrument of claim 3, wherein one or more or all of the positioning elements include one or more liner penetrating parts.

5. The instrument of claim 3, wherein two or more positioning elements are provided and wherein each positioning element includes a liner penetrating part towards its distal end.

6. The instrument of claim 5, wherein at least two parts of the curved edge provide, in use, the liner penetrating parts.

7. The instrument of claim 1, wherein a pair of positioning location supports extend to either side of the body element and the pair of positioning location supports provide two or more liner penetrating parts.

8. The instrument of claim 1, wherein a curved element and/or an edge is provided towards the distal end of the positioning element, and wherein the curved element and/or edge project away from the guide element towards a longitudinal axis of the guide channel.

9. The instrument of claim 1, wherein a distal end of a positioning location support provided by a positioning element and the distal end of a further positioning location support provided by a positioning element define a chord extending between them and the chord is closer to the longitudinal axis of the guide channel than a chord drawn between a first other part of the positioning location support and a first other part of the further positioning location support.

10. The instrument of claim 1, wherein one or more secondary positioning locations are provided by the one or more positioning elements and one or more of the secondary positioning locations abut the liner, in use.

11. A system, the surgical system comprising:
a surgical instrument, the instrument comprising:
a stem, the stem having a proximal end and having a distal end;
a handle portion, the handle portion being provided towards the proximal end of the stem; and
a guide element, the guide element being provided towards the distal end of the stem, wherein the guide element providing:
a body element defining a guide channel, the guide channel having a longitudinal axis and being open at both ends along the longitudinal axis; and
one or more positioning elements; and
the system further comprising:
one or more cutting tools; and
one or more elongate at least partially threaded elements.

12. A system according to claim 11, wherein the surgical systems further comprises one or more of:
an actuator for application of torque to the cutting tool;
one or more drill bits;
one or more self-tapping screw threads.

13. A system according to claim 12, wherein the guide channel of the surgical instrument is configured to receive the cutting tool with the cross-section of the guide channel being the cross-section of the cutting tool plus a clearance.

14. The system according to claim 13, wherein one or more or all of the positioning elements include one or more liner penetrating parts.

15. The system claim 11, wherein two or more positioning elements are provided and wherein each positioning element includes a liner penetrating part towards its distal end.
